(19) United States
(12) Patent Application Publication
(10) Pub. No.: US 2017/0065231 A1
(43) Pub. Date: Mar. 9, 2017

(54) NONINVASIVE BLOOD SUGAR MEASUREMENT DEVICE

(71) Applicant: GOLDENSUNDA TECHNOLOGY CO., LTD., New Taipei City (TW)

(72) Inventors: CHI-FA LIAO, New Taipei City (TW); CHIEN-TE LEE, New Taipei City (TW); HANG-KUANG HSU, New Taipei City (TW)

(21) Appl. No.: 14/845,283

(22) Filed: Sep. 4, 2015

(51) Int. Cl. A61B 5/00; A61B 5/04; A61B 5/145

(52) U.S. Cl. CPC A61B 5/7278; A61B 5/14532; A61B 5/14517; A61B 5/681; A61B 5/742; A61B 5/7405; A61B 5/0004; A61B 5/04; A61B 2562/04; A61B 2560/0223

(57) ABSTRACT

The noninvasive blood sugar measurement device contains a device body, a sensor, a processing module, and an error reduction module. The sensor is configured on a side of the device body for detecting a glucose level in perspiration. The processing module and the error reduction module are configured inside the device body. The processing module is data-linked with the sensor for calculating and converting a measured glucose level into a corresponding blood sugar level. The error reduction module is data-linked with the processing module for reducing a progressive error accumulated by the sensor from repeated detection of perspiration glucose level. The present invention therefore completely avoids invasive means such drawing blood and puncturing skin.

### BACKGROUND OF THE INVENTION

(a) Technical Field of the Invention

The present invention is generally related to the measurement of blood sugar, and more particular to a blood sugar measurement device without drawing blood or puncturing skin.

(b) Description of the Prior Art

Due to the widespread civil diseases, people are getting more and more concerned about their physical conditions and there is an increasing demand for health monitoring devices as visiting doctors is more difficult in the modern busy life.

Among the civil diseases, diabetes is probably the most common one. Diabetes has quite a few complications such as cardiovascular disease, chronic renal failure, diabetic retinopathy, diabetic microangiopathy, etc. These complications sometimes can be fatal and the monitoring of blood sugar level becomes very important for diabetic patients.

Existing blood sugar meters are mostly invasive ones, meaning their operation requires creating a wound in the skin (usually by a needle), collecting blood from the wood on a test strip, and processing the test strip by the blood sugar meter.

The invasive blood sugar meters have a number of disadvantages.

Firstly, the invasive blood collection process usually causes uncomfortableness. For diabetic patients, the wound may not easily heal.

Secondly, every test requires a new test strip which may be a financial burden to some patients.

### SUMMARY OF THE INVENTION

Therefore the present invention provides a noninvasive blood sugar measurement device.

A major objective of the present invention is to detect a glucose level in human perspiration through a sensor, convert the detected glucose level to a corresponding blood sugar level, and reduce a progressive error by the sensor. The present invention therefore completely avoids invasive means such drawing blood and puncturing skin.

The noninvasive blood sugar measurement device contains a device body, a sensor, a processing module, and an error reduction module. The sensor is configured on a side of the device body for detecting a glucose level in perspiration. The processing module and the error reduction module are configured inside the device body. The processing module is data-linked with the sensor for calculating and converting a measured glucose level into a corresponding blood sugar level. The error reduction module is data-linked with the processing module for reducing a progressive error accumulated by the sensor from repeated detection of perspiration glucose level.

A user then wears the noninvasive blood sugar measurement device so that the sensor contacts the user's perspiration. The different levels of glucose in the perspiration would lead to different electrical characteristics. The sensor therefore detects a voltage variation, and the processing module then converts the voltage variation into a corresponding blood sugar level based on a ratio between the blood sugar and glucose. The sensor may contain some residual glucose that will interfere the accurate detection of blood sugar level. The error reduction module uses electrical calibration or compensation to remove or reduce such kind of error. As such the noninvasive blood sugar measurement device is able to constantly produce accurate blood sugar reading without drawing blood or puncturing skin.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1A is a perspective diagram showing a noninvasive blood sugar measurement device according to an embodiment of the present invention.

FIG. 1B is an enlarged diagram showing a sensor on the noninvasive blood sugar measurement device of FIG. 1A.

FIG. 2 is a functional block diagram showing the noninvasive blood sugar measurement device of FIG. 1A.

FIG. 3 is a schematic diagram showing the noninvasive blood sugar measurement device of FIG. 1A worn on a user's wrist

FIG. 4 is a schematic diagram showing the noninvasive blood sugar measurement device of FIG. 3 displaying a measured blood sugar level.

FIG. 5 is a schematic diagram showing the noninvasive blood sugar measurement device of FIG. 3 transmitting a measured blood sugar level to a smart phone.

FIG. 6 is a perspective diagram showing another a noninvasive blood sugar measurement device according to another embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

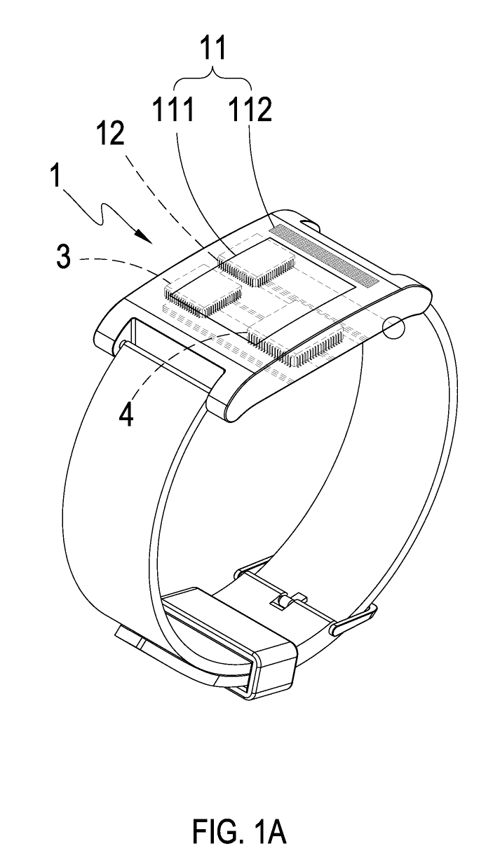

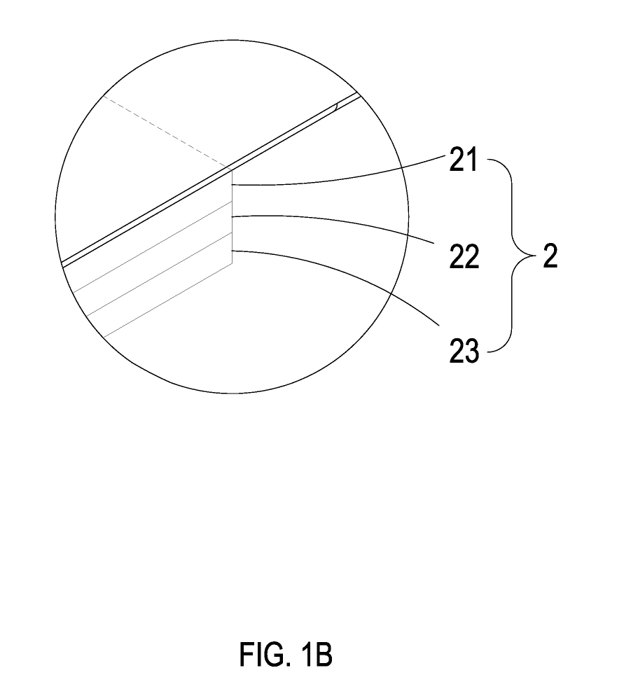

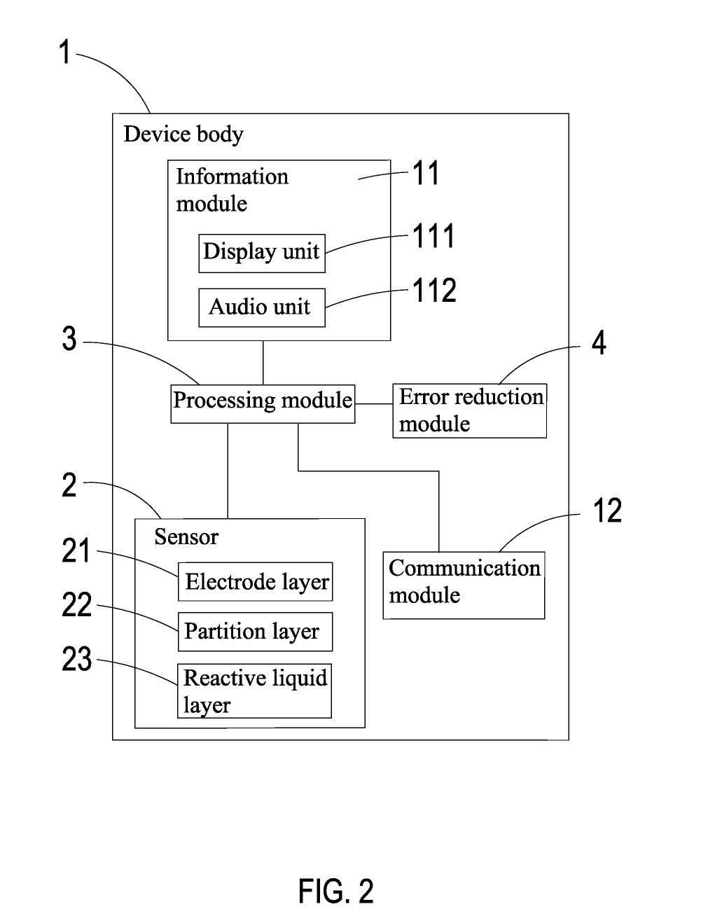

As shown in FIGS. 1A to 6, a noninvasive blood sugar measurement device according to an embodiment of the present invention contains following components.

There is a device body 1.

A sensor 2 is configured on a side of the device body 1 for detecting a glucose level in perspiration. The sensor 2 contains an electrode layer 21 configured on the side of the device body 1, a partition layer 22 covering the electrode layer 21, and a reactive liquid layer 23 on the partition layer 22 that contacts user skin.

A processing module 3 is configured inside the device body 1 data-linked with the sensor 2 for calculating and converting a measured glucose level into a corresponding blood sugar level.

An error reduction module 4 configured inside the device body 1 data-linked with the processing module 3 for eliminating a progressive error accumulated by the sensor 2 from repeated detection of perspiration glucose level.

In the present embodiment, the device body 1 is provided on a wristband, and the noninvasive blood sugar measurement device further contains an information module 11 configured on another side of the device body 1 for delivering information to the user visually and audibly. The information module 11 is data-linked to the processing module 3, and contains a display unit 111 and an audio unit 112 so as to present information such as a measured blood sugar level to the user on a screen of the display unit 111 or through a speaker of the audio unit 112.

The noninvasive blood sugar measurement device may further contain a communication module 12 configured inside the device body 1. The communication module 12 is data-linked with the processing module 3 for transmitting information to another information device.

Preferably there are three or more sensors 2 so as to avoid signal loss and interference. The electrode layer 21 contains a conductive material such as a metallic material, and can have a circular or rectangular or another shape as long as the electrode layer 21 is able to provide adequate surface area. The partition layer 22 contains a fabric or a gel of semi-conductivity for covering the electrode layer 21 to achieve separation, to avoid short circuit, and to prevent the electrode layer 21 from directly contacting user skin. The reactive liquid layer 23 contains a glucose enzyme so that, when the reactive liquid layer 23 contacts user skin, the glucose enzyme reacts with the glucose in perspiration, leading to variation in electrical voltage or current.

The operation principle of the present invention is as follows. Human perspiration always contains a certain amount of glucose. When the blood sugar level in human body is higher, there is more glucose in the perspiration. The different levels of glucose in the perspiration, when the glucose is reduced, would lead to different electrical characteristics. According to the present invention, the reactive liquid layer 23 of the sensor 2 therefore would release different amounts of electrons after reacting with the glucose of the perspiration. As the electric charge of the reactive liquid layer 23 increases, the sensor 2 would detect a voltage variation. Based on the ratio between the blood sugar and glucose, the processing module 3 then converts the voltage variation into a corresponding blood sugar level, without drawing blood or puncturing skin.

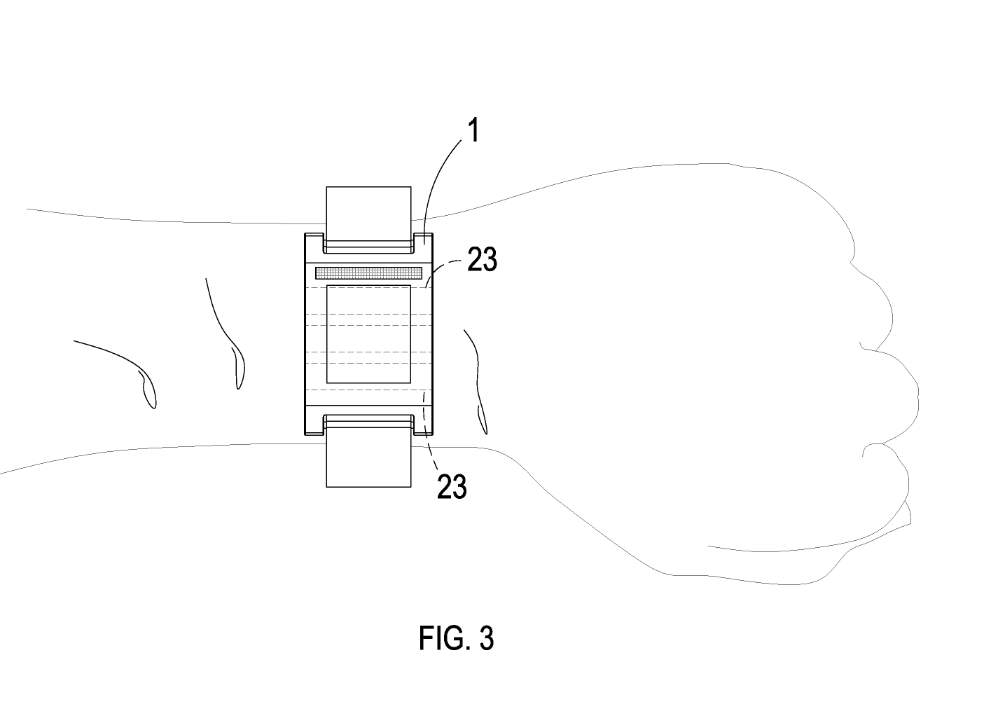

As shown in FIG. 3, the noninvasive blood sugar measurement device is worn around a user's wrist. The reactive liquid layer 23 on the device body 1 therefore is able to contact the user's skin to measure the user's blood sugar level.

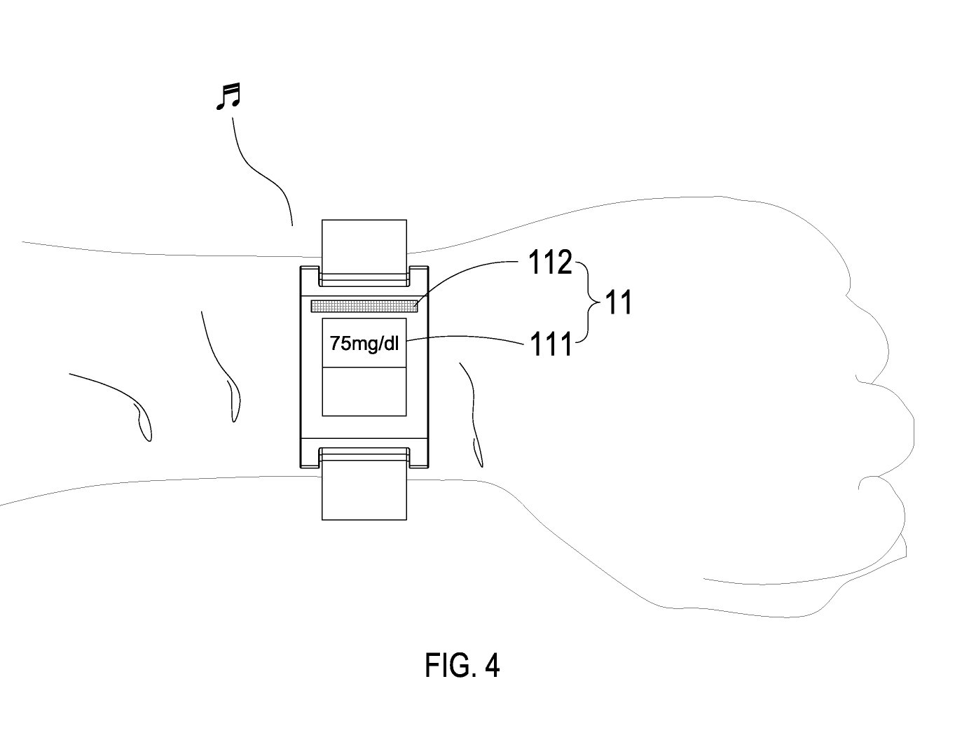

As shown in FIG. 4, after the processing module 3 obtains the blood sugar level, the result is presented to the user visually through the display unit 111 and/or audibly through the audio unit 112.

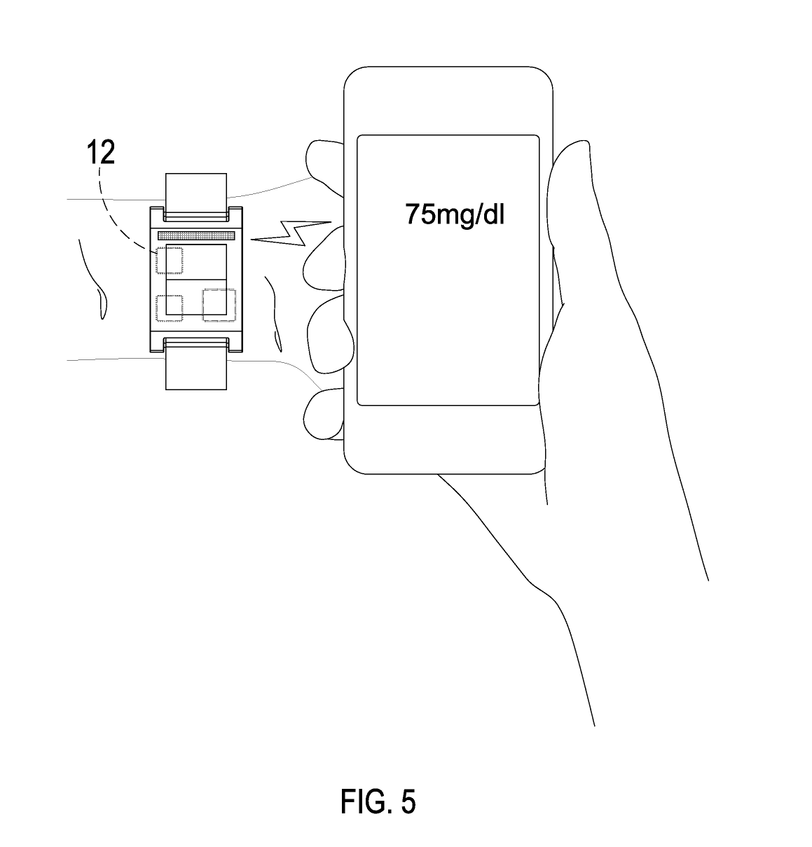

As shown in FIG. 5, the measured blood sugar level can also be transmitted to another information device via the communication module 12. For example, the communication module 12 can be a Bluetooth transceiver that, after pairing with a smart phone, the measured blood sugar level can be read from the smart phone's big screen.

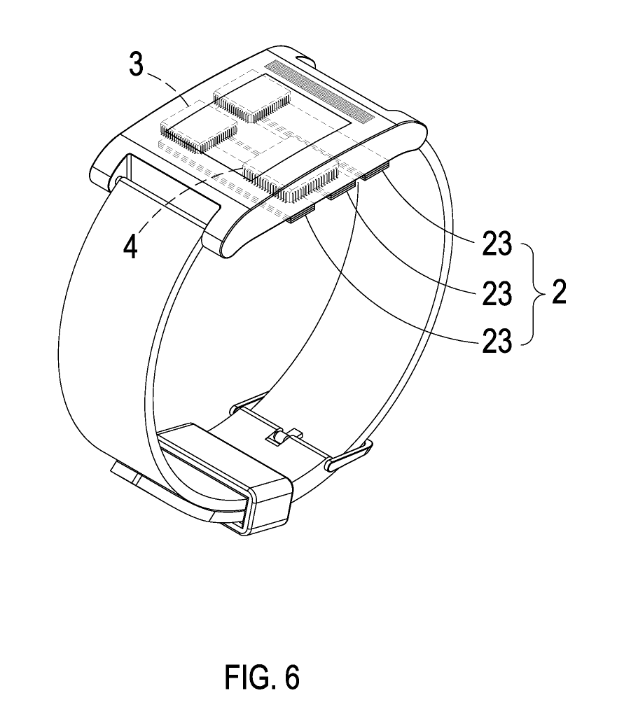

As shown in FIG. 6, the present invention may further contain an error reduction module 4. When the noninvasive blood sugar measurement device has put to use for a period of time, the reactive liquid layer 23 may contain some residual glucose that will interfere the accurate detection of blood sugar level. In the worst scenario, the noninvasive blood sugar measurement device may produce some high readings even without contacting perspiration. In order obviate this problem, the error reduction module uses electrical calibration or compensation to remove or reduce such kind of error so that the processing module 3 is able to constantly produce accurate blood sugar reading.

In summary, the gist of the present invention lies in the adoption of a sensor 2 to detect glucose level in perspiration and a processing module 3 to convert the detected glucose level into a corresponding blood sugar level, therefore completely avoiding invasive means such drawing blood and puncturing skin.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A noninvasive blood sugar measurement device comprising:
a device body;
at least a sensor configured on a side of the device body for detecting a glucose level in perspiration;
a processing module configured inside the device body data-linked with the sensor for calculating and converting a measured glucose level into a corresponding blood sugar level; and
an error reduction module configured inside the device body data-linked with the processing module for reducing a progressive error accumulated by the sensor from repeated detection of perspiration glucose level.

2. The noninvasive blood sugar measurement device according to claim 1, further comprising a wristband where the device body is configured on a side of the wristband.

3. The noninvasive blood sugar measurement device according to claim 1, further comprising an information module configured on another side of the device body data-linked to the processing module for delivering information to a user.

4. The noninvasive blood sugar measurement device according to claim 3, wherein the information module comprises one of a display unit and an audio unit so as to present information to the user visually or audibly.

5. The noninvasive blood sugar measurement device according to claim 1, further comprising a communication module configured inside the device body data-linked with the processing module for transmitting information to another information device.

6. The noninvasive blood sugar measurement device according to claim 1, wherein the sensor comprises an electrode layer configured on the side of the device body, a partition layer covering the electrode layer, and a reactive liquid layer on the partition layer for contacting user skin; the reactive liquid layer absorbs electrons released from reducing perspiration glucose and accumulates electric charges; and the sensor as such manifests a voltage variation.

7. The noninvasive blood sugar measurement device according to claim 6, wherein there are at least three sensors so as to avoid signal loss and interference.

8. The noninvasive blood sugar measurement device according to claim 6, wherein the processing module converts the voltage variation to a corresponding blood sugar level according to a relationship between perspiration glucose and blood sugar.

9. The noninvasive blood sugar measurement device according to claim 6, wherein the error reduction module uses electrical calibration to reduce error caused by residual glucose in the reactive liquid layer so that the processing module is able to produce accurate blood sugar reading.
